(19) United States
(12) Patent Application Publication
(10) Pub. No.: US 2024/0269669 A1
(43) Pub. Date: Aug. 15, 2024

(54) MICROCHANNELS WITH ELECTRODE SENSORS

(71) Applicant: Coagulo Medical Technologies, Inc., Auburndale, MA (US)

(72) Inventors: Galit H. Frydman, Boston, MA (US); Justin David MORSE, Providence, RI (US); Leonid SILVERMAN, Cranston, RI (US)

(21) Appl. No.: 18/568,824

(22) PCT Filed: Jun. 9, 2022
(86) PCT No.: PCT/US2022/032914

§ 371 Date: Dec. 10, 2023

Related U.S. Application Data
(60) Provisional application No. 63/209,728, filed on Jun. 11, 2021.

(51) Int. Cl. B01L 3/00; G01N 27/02; G01N 33/49; G01N 33/86

(52) U.S. Cl. CPC B01L 3/502707; G01N 27/026; G01N 33/4905; G01N 33/86

(57) ABSTRACT

Various embodiments of the present invention provide microchannel and electrode configurations by which uniform electric current density within a microchannel is achieved. The invention provides this and other advantages.

### FIELD OF THE INVENTION

The present invention relates to devices and methods for the generation of electric current within a microchannel using uniplanar electrodes, wherein the electric current density is sufficiently uniform to provide sensitive detection and measurement of electrical properties within the microchannel. Accordingly, various embodiments of the invention provide microchannel and electrode configurations that allow for sensitive measurement of changes in electrical properties (for example, resistance, capacitance, inductance, impedance, permittivity) within and throughout the medium in the microchannel. The devices and methods described herein can be used for various applications, including for the evaluation of coagulation in a blood sample.

### BACKGROUND

The use of microfluidics for diagnostics and other applications is becoming increasingly common. The use of electrodes (e.g., electrode sensors) within the microfluidic device for measuring changes in electrical properties as a read-out is a useful approach, as it tends to be robust and low-cost.

There are many factors that affect the sensing capabilities of an electrode sensor within a microchannel. The shape, size, orientation, and material of the electrode, as well as features of the microchannel, are factors that affect the level and distribution of electric current within the microchannel, and each factor in turn may affect the ability of the electrodes to sense changes within the medium contained in the microchannel, whether that medium is a liquid, a semi-solid, or air.

There is a need in the art for microfluidic devices with electrodes that provide an electric current within a microchannel, wherein the electric current is both sufficiently high in density and sufficiently even in distribution, such that the electrodes are able to sensitively detect changes in an electrical property within the medium in the microchannel, including changes that are small in magnitude and/or that may occur at different locations within the microchannel (including, for example, locations that are not near an electrode). Further, there is a need in the art to provide such microchannels with electrodes that are uniplanar, as uniplanar electrodes can be less complex to manufacture compared to multiplanar electrodes and may allow for more types of (and in some instances less costly) fabrication techniques.

### SUMMARY OF THE INVENTION

Embodiments of the invention provide a microchannel comprising uniplanar electrodes. In certain embodiments, the microchannel has a hexagonal geometry with six edges and comprises two electrodes in a uniplanar configuration, wherein the two electrodes are oriented to be opposite one another on the same plane within the microchannel. In some embodiments, the two electrodes each comprise a rectangular portion that is oriented to be parallel to one of the microchannel's six edges and parallel to the rectangular portion of the other electrode's rectangular portion. In further embodiments, one or more of the following features is present: the electrode height:channel height ratio is at least 0.1; the electrode spacing:channel width ratio is at least 0.5; the two electrodes each comprise two circular regions, each circular region located at an electrode end.

In certain embodiments, the ratio of electrode height:channel height of such microchannel is at least 0.1, at least 0.3, at least 0.5, at least 0.6, at least 0.8, or at least 0.9. In certain embodiments, the electrode height:channel height ratio is within the range of 0.3 to 0.5. In some embodiments, the electrode height:channel height ratio is 0.5 or greater.

In certain embodiments, the ratio of electrode spacing (the distance between the electrodes, as measured between the rectangular portions of the electrodes-see FIG. 6A(ii) (electrode spacing)) to channel width is 0.5 or greater. In further embodiments, the electrode spacing:channel width ratio is 0.6 or greater, or is 0.7 or greater. In certain embodiments, the electrode spacing:channel width ratio is 0.8 or greater. In further embodiments, the electrode spacing:channel width ratio is 0.9 or greater.

In certain of the embodiments where each of the two electrodes comprises a circular region at each of its ends, each circular region has a diameter that is within the range of 300 microns to 600 microns. In further embodiments, each circular region has a diameter that is 500 microns. For each electrode, an electric via may be located within one of the circular regions.

In certain embodiments, the microchannel volume is less than or equal to 2.5 microliters. In further embodiments, the microchannel volume is less than or equal to 2.0 microliters.

In certain embodiments, the microchannel is fabricated using one or more minimally thrombogenic materials (such as, for example, polyimide). The microchannel may be fabricated such that the interior surface of the microchannel (and the surface that contacts the sample) is minimally thrombogenic.

In certain embodiments, the electrodes comprise a thrombogenic material. For example, the electrodes may be made with a thrombogenic material and/or may contain a coating, wherein the coating comprises a thrombogenic material (such a coating may be referred to as a thrombogenic coating). Thrombogenic materials that may be used in a coating include, for example, metals such as gold, silver, and platinum. When such coating is used, the surface of the electrode is thrombogenic.

Embodiments of the present invention also provide a hexagonal microchannel comprising two electrodes having the same geometry and oriented to be opposite one another on the same plane within the microchannel. Each electrode may comprise a rectangular or linear portion that is parallel to one of the microchannel's six edges and that is located no more than 500 microns from the edge to which it is parallel. In addition, in certain embodiments the distance between electrodes (the distance between the electrode's rectangular or linear portions) is at least 4,000 microns, and in further embodiments the length of each edge is no longer than 4,000 microns. In certain embodiments, the ratio of electrode height:channel height of such microchannel is at least 0.1, at least 0.3, at least 0.5, at least 0.6, at least 0.8, or at least 0.9; and in certain embodiments, the electrode height:channel height ratio is 0.5 or greater. In further embodiments, the microchannel may be fabricated with a minimally thrombogenic material, such that the interior surface of the microchannel (and the surface that contacts the sample) is minimally thrombogenic. In additional embodiments, the electrodes may be made with a thrombogenic material and/or may contain a coating, wherein the coating comprises a thrombogenic material. In certain embodiments, the microchannel volume is less than or equal to 2.5 microliters; in further embodiments, the microchannel volume is less than or equal to 2.0 microliters.

The microchannel-electrode configurations provided herein provide a uniform distribution of electric current throughout the microchannel when a voltage is applied. For example, in certain embodiments, when an excitation voltage is applied to a microchannel containing medium with electrical (e.g., conductive) properties similar to uncoagulated blood, the electric current density across at least 70% of the area of a cross-sectional plane of the microchannel does not vary by more than 20%. In further embodiments, the electric current density across at least 80% of the area of a cross-sectional plane of the microchannel does not vary by more than 20%. In addition, in certain embodiments, the electric current density of at least 80% of the microchannel volume does not vary by more than 20%.

The present invention relates to these and other important aspects, as described herein.

### BRIEF DESCRIPTION OF THE DRAWINGS

FIGS. 1A-1C provide schematics of electric current flowing through blood and show how electrodes can detect changes in electrical impedance. Electrical impedance may result from ohmic resistances as well as capacitive and inductive reactances of components of the blood sample. For example, for FIG. 1A, the blood contains red blood cells: Cm is the capacitance of the red blood cell membrane, Ri is the internal resistance of the red blood cell, and Rp is the resistance of the plasma. FIG. 1B and FIG. 1C are schematic illustrations showing current flow through a sample of blood containing red blood cells and white blood cells. FIG. 1B depicts non-coagulated blood, where the cells are separated. FIG. 1C depicts coagulated blood, where the red blood cells and white blood cells are clustered together in aggregates. In both FIG. 1B and FIG. 1C, the solid arrows represent low frequency current, and the dashed arrows represent high frequency current. These schematics illustrate how low and high frequencies can be used to detect and measure changes in electrical impedance (or other electrical property) when, for example, a voltage is applied to a sample.

FIGS. 2A-2D provide various configurations of microchannels containing electrodes, with various electrode orientations and geometries and microchannel geometries, and show simulated electric current generation within the microchannels. Simulations conducted on the various configurations demonstrate how electric current distribution within a microchannel can vary as a result of microchannel geometry and electrode geometry and placement. None of these configurations provides uniform electric current density throughout the microchannel.

FIGS. 3A-3C provide two configurations of microchannels containing electrodes, and show simulated electric current generation within each microchannel configuration.

FIGS. 4A-4B provide schematics showing simulated current generation and blood clot detection in two different configurations: FIG. 4A depicts an interdigitated configuration, and FIG. 4B depicts a two-electrode configuration.

FIGS. 5A-5F provide clot detection data for the six configurations described in Example 4. For each graph, impedance (ohms, y-axis) is plotted over time (seconds, x-axis).

FIGS. 6A-6B provide simulations of electric current in microchannels of various channel heights, using a two-electrode microchannel configuration, and illustrate how the distribution of current becomes less uniform as microchannel height increases.

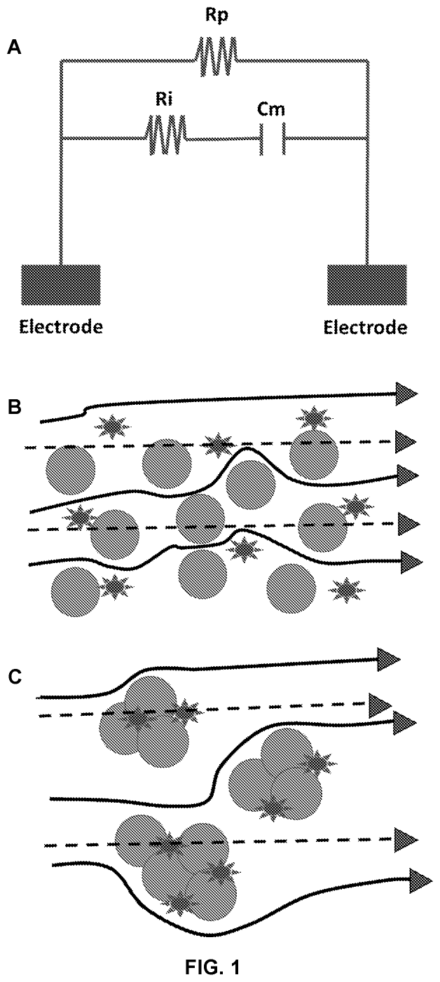

### DETAILED DESCRIPTION

Microchannel devices with electrodes can be used for biological fluid analysis, as well as for environmental analysis, such as to test water quality. The devices may contain sensors separate from the electrodes, or the electrodes themselves may operate as sensors; in either case, the sensors detect electrical changes (changes in one or more electrical properties such as, e.g., admittance, conductivity, impedance, resistance, dielectric permittivity) in the channel medium that occur as a result of, e.g., a chemical reaction or a physical change in the medium or both. The detection and measurement of electrical changes in a sample is sometimes referred to as impedance spectroscopy or dielectric spectroscopy. In the case of assessing coagulation of a sample, impedance spectroscopy can involve evaluating impedance changes over a range of frequencies, when looking at, e.g., whole blood, which contains both plasma and blood cells.

In certain embodiments described herein, the electrodes operate as sensors for measuring changes in electrical properties in the microchannel medium.

In some embodiments, the electrodes within the microchannel may serve additional functions. For example, in certain cases involving chemical reactions, the electrodes themselves can play a role in instigating the chemical reaction. In certain cases, an electrode may comprise a material (such as, e.g., in the form of a coating on the electrode) that plays a part in the chemical reaction.

In addition, electrodes may be functionalized with one or more agents. Such agents can serve several roles, including by participating in a chemical reaction, by localizing the reaction to the sensor region, or both. For example, electrodes may be functionalized with proteins, such as antibodies or antigens, for direct or indirect ELISA purposes.

Electrodes within a microchannel may also promote mixing of the medium and other contents within the microchannel. For example, the electrodes, by providing a physical obstacle to the flow of the medium within the microchannel, may disturb the flow of the medium in such a way so as to promote mixing. See generally Nguyen et al., Recent Advances and Future Perspectives on Microfluidic Liquid Handling, Micromachines 8: 186 (2017); Ward and Fan, Mixing in microfluidic devices and enhancement methods, J. Micromech. Microeng., 25, 094001 (2015). In addition or alternatively, the applied voltage can produce a mixing effect. E.g., Song et al., Chaotic mixing in microchannels via low frequency switching transverse electroosmotic flow generated on integrated microelectrodes, Lab on a Chip, 10: 734-740 (2010). In certain embodiments, depending on the mechanism of mixing desired, a lower electrode height:channel height ratio may be preferred.

Microfluidic channels with electrodes can be used to measure a variety of changes in blood, or in one or more components of blood. In embodiments of the present invention, microchannel-electrode configurations are provided that are particularly useful for detecting and evaluating coagulation, which is a process that results in the formation of a cross-linked fibrin clot. In addition, for whole blood, the devices and methods described herein can be used to detect and/or measure changes in circulating cells, such as changes in red blood cell membrane capacitance. In other embodiments, the devices and methods can be used to detect and/or measure changes in platelet morphology and activation (e.g., attachment and aggregation).

For example, measuring changes in electrical impedance (or in one or more other electrical properties, such as resistance, capacitance, inductance, and permittivity) may be used for clot detection in microfluidic channels. In some examples, the electrodes may be placed on glass or other substrate that activates the coagulation contact pathway. Placing electrodes on a coagulation contact pathway-activating substrate (e.g., glass) will promote localization of clot formation on such substrate and therefore near where the electrodes are located; promoting the formation of the clot around the electrodes may improve clot detection by the electrodes. Accordingly, in certain embodiments, the electrodes can be placed directly on a thrombogenic substrate.

The substrate onto which the electrodes are placed may be the same as or different from the material (or materials) that forms the surface of the microchannel. For example, in some embodiments, the electrode is positioned to be directly on the floor of the microchannel, and the floor of the microchannel is the substrate onto which the electrode is placed; in other embodiments, there is an additional material (a material that differs from the floor of the microchannel) that serves as the substrate onto which the electrode is placed.

In some embodiments, the electrodes may be placed on a minimally thrombogenic substrate (such as, e.g., Kapton®) and the electrodes themselves are the most thrombogenic surface in the microchannel. Similarly, in certain embodiments where the substrate onto which the electrodes are placed is also thrombogenic, the electrodes can be more thrombogenic than the substrate (and all other surfaces within the microchannel), such that the electrodes are the most thrombogenic surface in the microchannel. In such embodiments where the electrodes provide the most thrombogenic surface that is in contact with the sample, the electrodes help localize clot formation near the sensors (or on the sensors, in embodiments where the electrodes also operate as sensors), which can further improve the device's ability to detect clot formation. Assessing coagulation using such devices can be performed without the addition of activators, as the electrodes serve as an activator of the intrinsic coagulation pathway. Alternatively, activators (such as, e.g., glass microspheres or ground glass, kaolin, silica, activated coagulation factors, phospholipids, tissue factor, etc.) may be added. Electrodes can be made to be thrombogenic by, for example, including a coating (such as gold or other metal coating) on the electrode. See generally Hulander et al., Blood interactions with noble metals: coagulation and immune complement activation, ACS Appl. Mater. Interfaces 2009 May;1(5):1053-62.

There are few descriptions of coagulation detection in microchannels formed entirely or partially of materials that are minimally thrombogenic (e.g., materials such as various plastics, including but not limited to polyimide). One example is the use of planar electrodes positioned on parallel planes of a channel. See, e.g., Kucukal et al., Monitoring blood coagulation using a surface-functionalized microfluidic dielectric sensor, 2017 IEEE 12th International Conference on Nano/Micro Engineered and Molecular Systems (NEMS), 752-755 (polyethylene glycol-coated microchannel); Maji et al., Assessment of whole blood coagulation with a microfluidic dielectric sensor, J. Thromb. Haemost. 16: 2050-2056 (2018) (polymethyl methacrylate (PMMA) plastic substrate). In another example, the electrodes are placed in their own channel (separate from the channel containing the sample), and the current must pass through a microtube for the detection of clot formation.

Embodiments of the present invention provide microchannels with electrodes in a uniplanar configuration. Using uniplanar electrode configurations provides several advantages. For example, using uniplanar (as opposed to multiplanar) electrodes may allow for more types of fabrication techniques and materials, including more ways for integrating the electrodes into the microchannel. In particular, using uniplanar electrodes permits the use of a printed circuit board (PCB) for forming one or more microchannel surfaces, as a PCB can be used as the substrate onto which the uniplanar electrodes are placed.

In further embodiments, the uniplanar electrodes can be placed on a minimally thrombogenic substrate. Using a minimally thrombogenic substrate can provide similar advantages with respect to fabrication of the microchannel-electrode system. For example, with respect to using a PCB, the PCB can be made of a minimally thrombogenic material, such as, e.g., polyimide. Minimally thrombogenic materials include materials that generally have a low tendency to produce a clot when in contact with blood, when compared to other materials considered to be thrombogenic (materials such as glass and gold, for example). Minimally thrombogenic materials include materials that may be called non-thrombogenic. It should be understood that there are various techniques that can be used to place electrodes on a substrate, such as, for example, plating, printing, electron spray deposition, sputtering, annealing, and adhering (via an adhesive or other method).

In certain embodiments, the material used for the surface of the substrate, and the material used for the interior surface of the microchannel (if different from the substrate or from the substrate's material), are minimally conductive (minimally conductive materials include materials that may be called non-conductive). The conductivity of a material refers to the material's ability to conduct electric current. In embodiments described herein, a material in contact with the sample in the microchannel (e.g., the material used for the interior surface of the microchannel) has low electrical conductivity compared to the conductivity of the sample, such that a negligible amount of current flows through the material. Accordingly, minimally conductive materials include materials (such as, c.g., polyimide, polycarbonate, and acrylic) that are less conductive compared to materials such as gold and blood, for example (see Table 2).

Accordingly, in some embodiments, the present invention provides uniplanar electrodes in a single microfluidic channel. Such embodiments further provide microchannel-electrode configurations that produce a uniform distribution of electric current. In the examples described herein, the use of uniplanar electrodes placed on a minimally thrombogenic and minimally conductive substrate (e.g., a substrate comprising or coated with a plastic, such as, c.g., polyimide) produces a uniform electric current density throughout a microchannel, and allows for successful detection of blood clot formation throughout the microchannel (as opposed to only at certain locations or portions within the microchannel). While some of the examples described herein relate to detection of coagulation in a sample, the microchannels described herein are useful for any application where a uniform electric current within a microchannel is desirable (e.g., for agglutination and/or aggregometry). For example, the various microchannel-electrode configurations described herein can be used for any impedimetric (or conductometric, etc.) analysis of a sample (such as for detection of coagulation but also for the detection of other changes in a sample that affect an electrical property) as well as for treating a sample with an electric current.

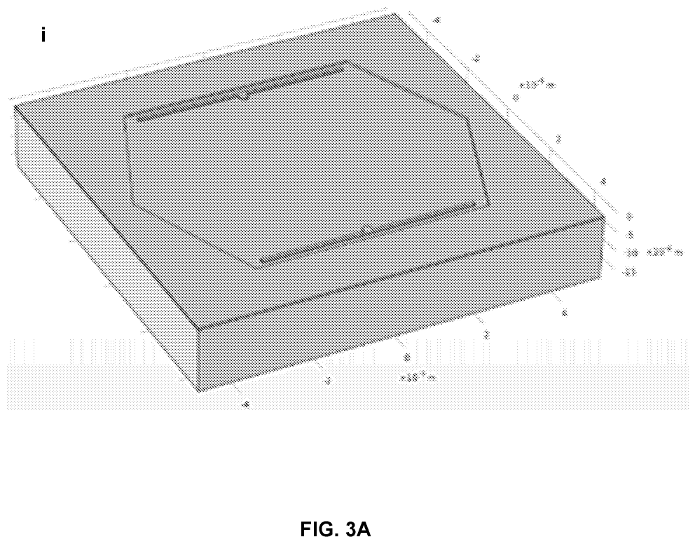

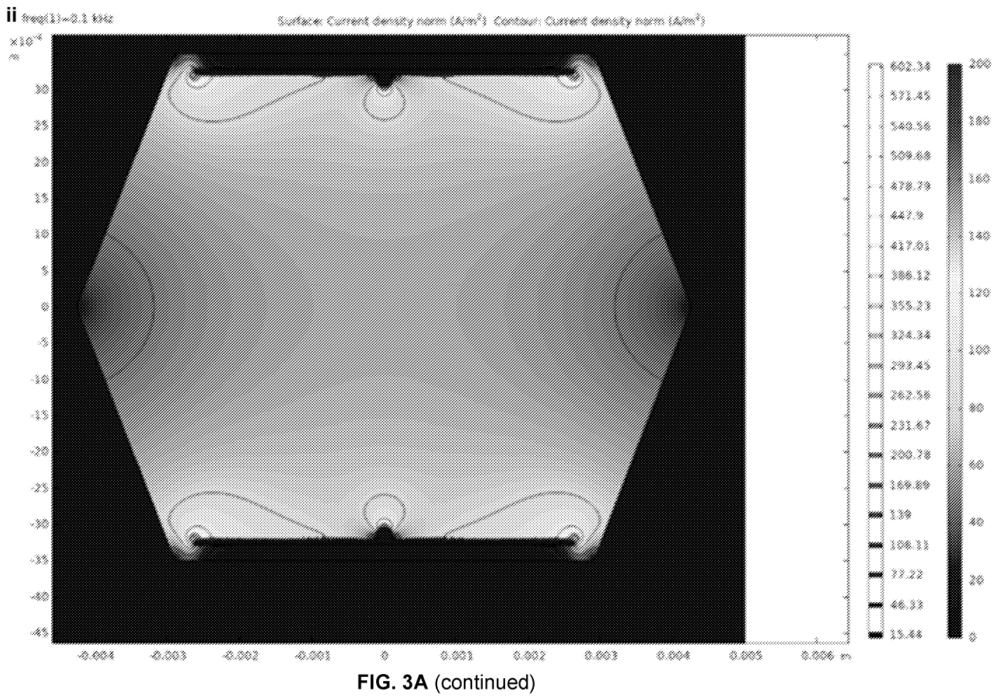

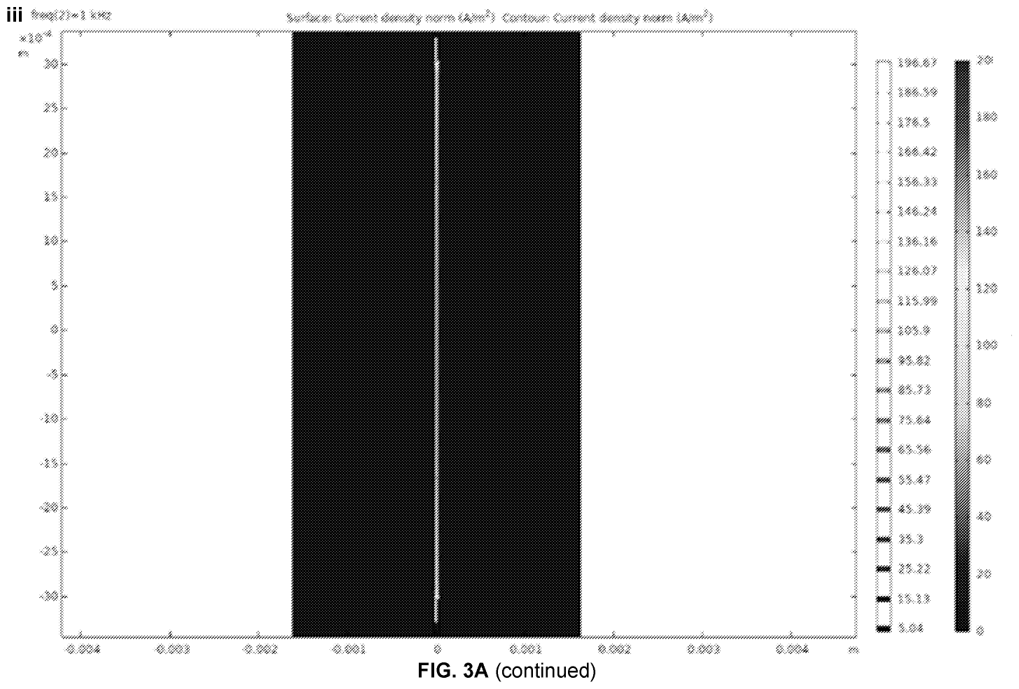

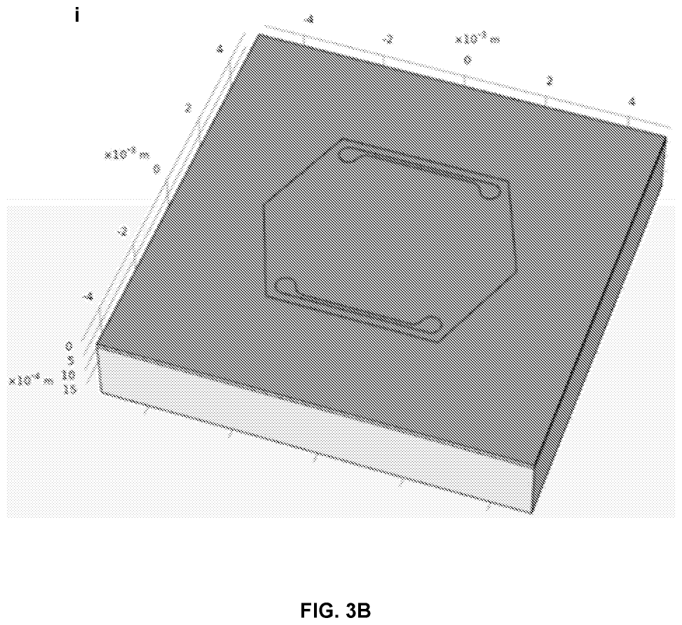

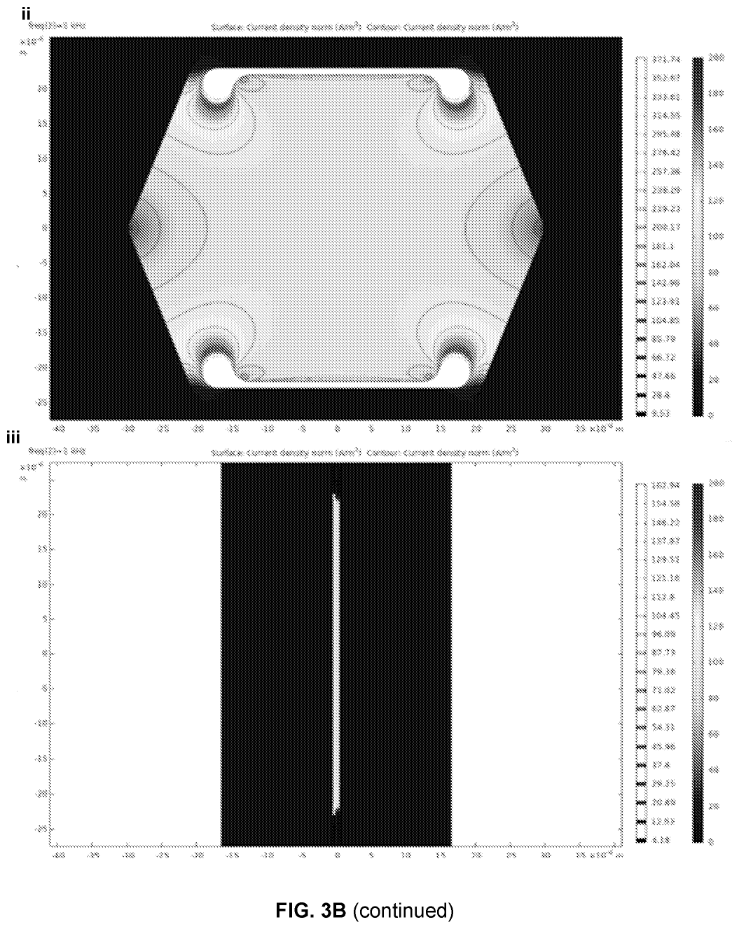

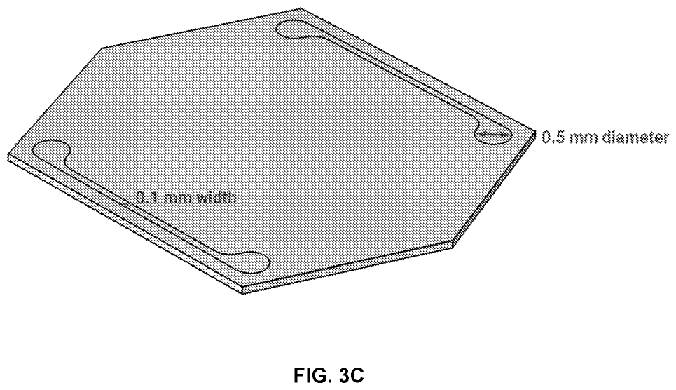

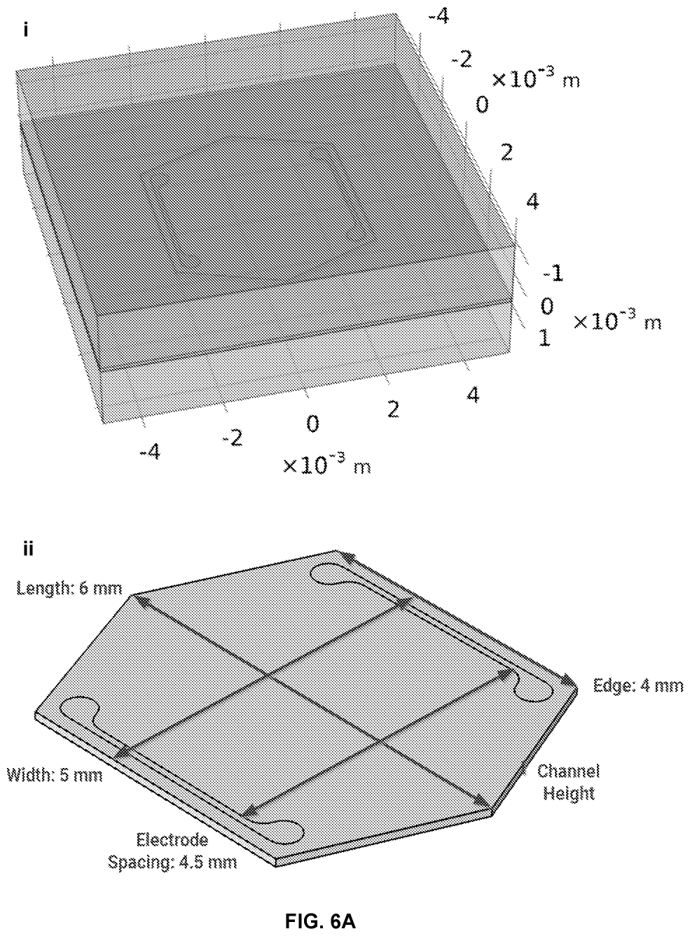

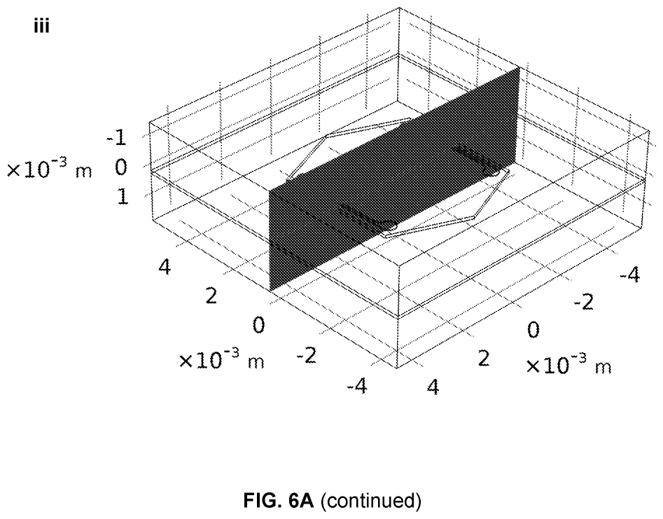

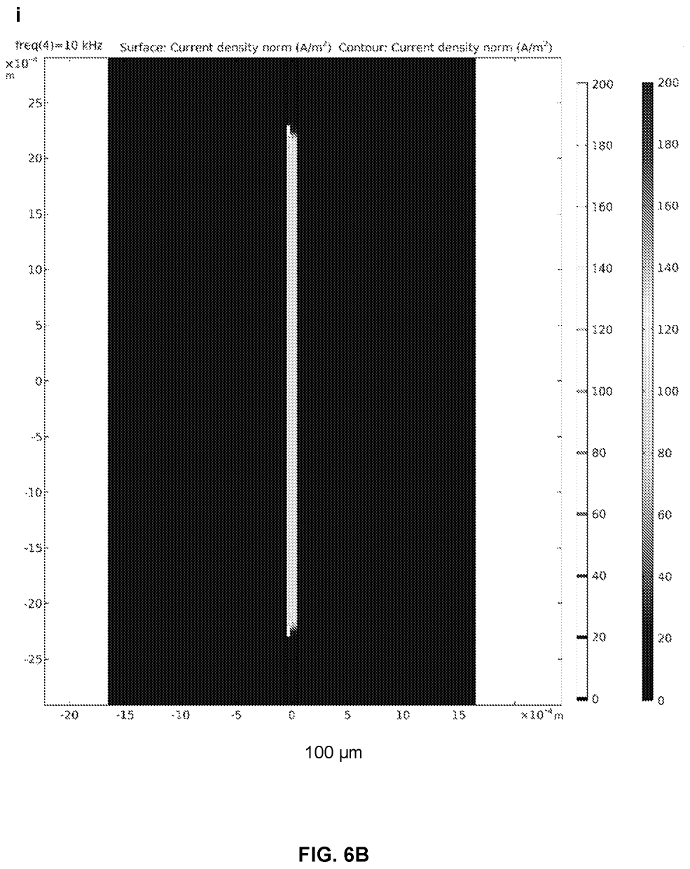

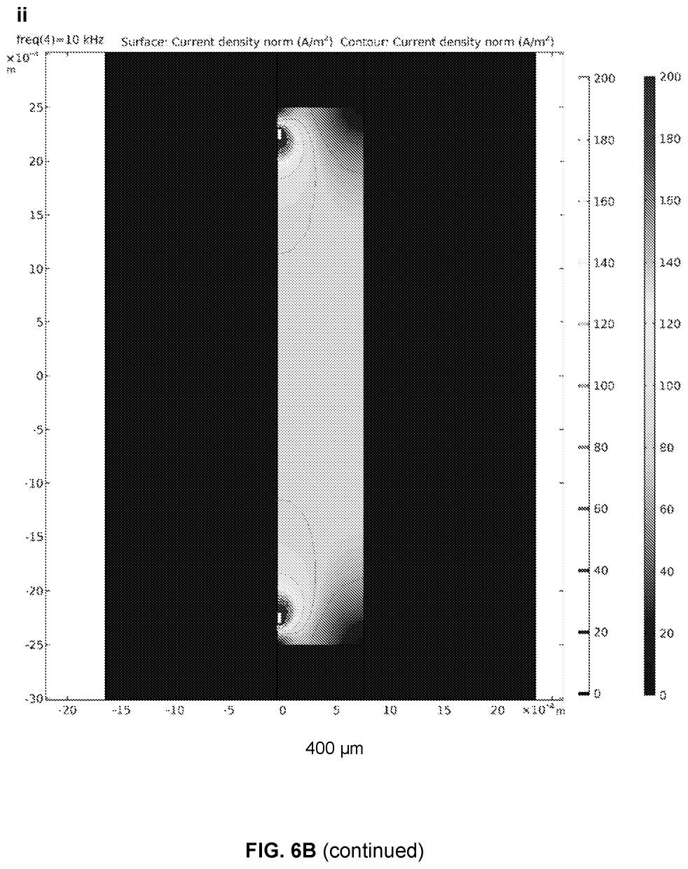

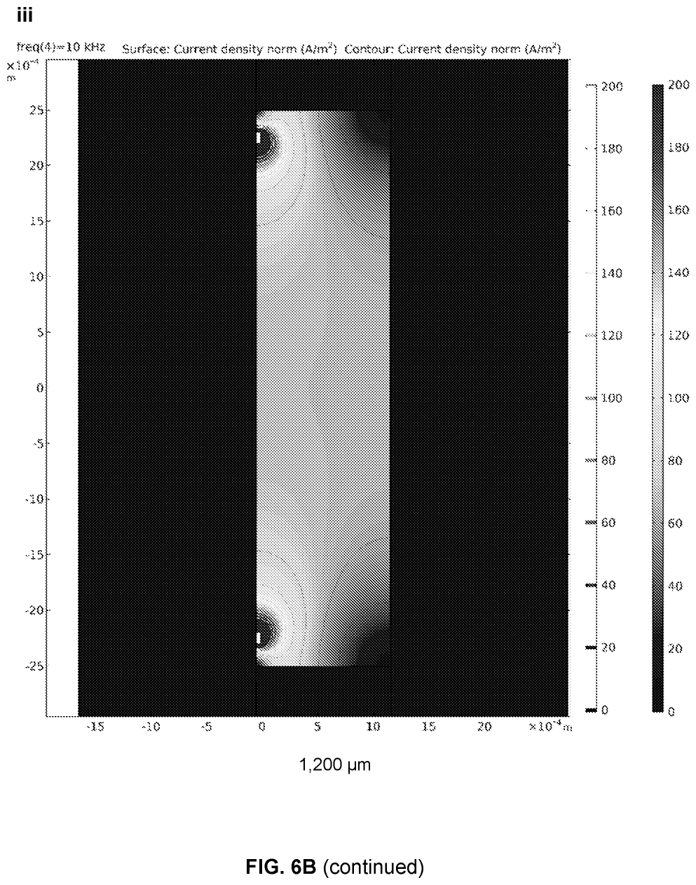

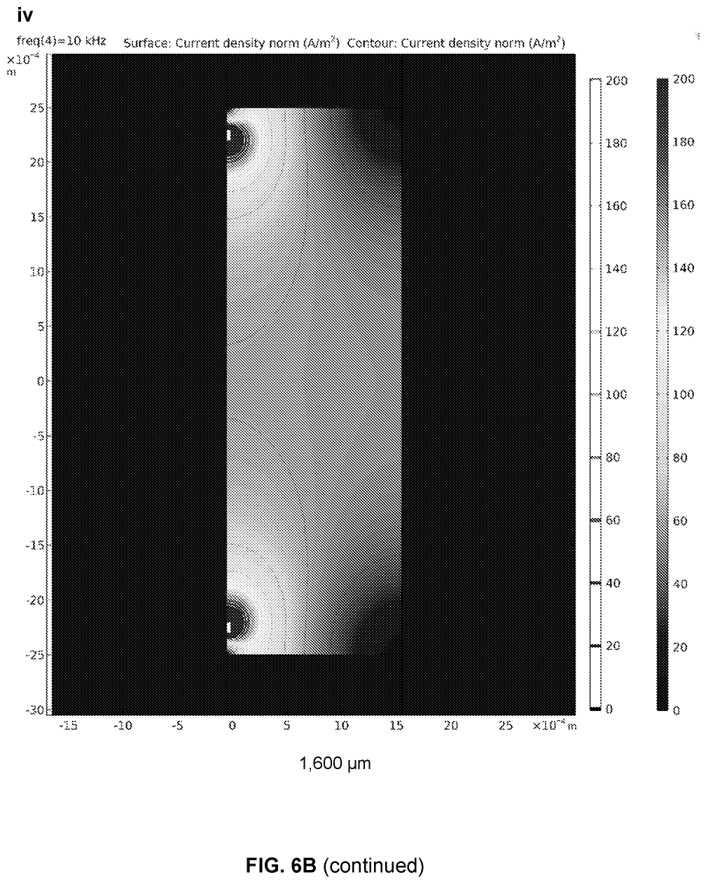

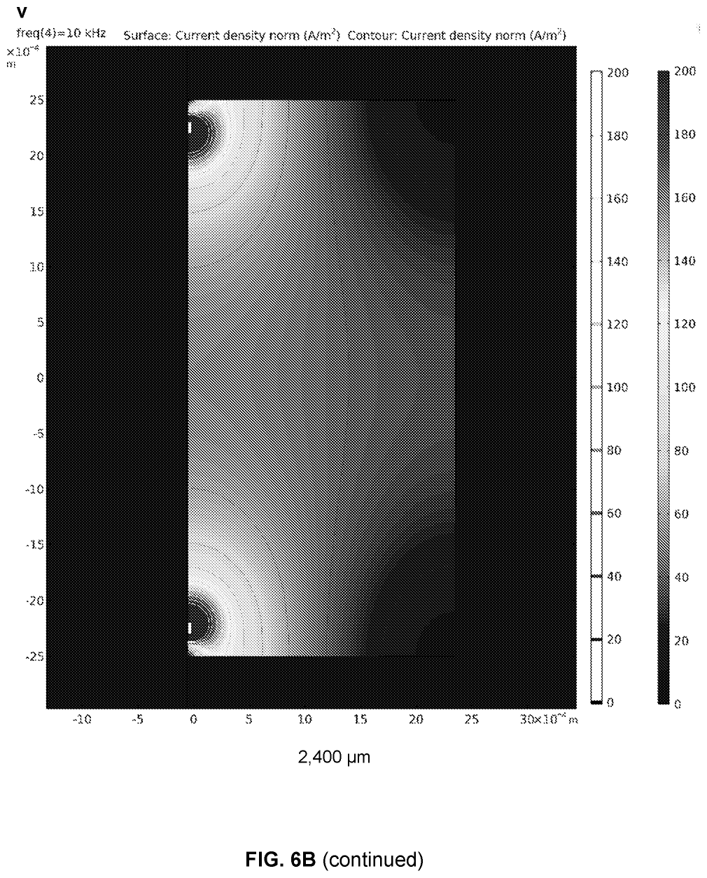

In addition to providing uniform electric current distribution, the microchannels described herein can be used for small sample volumes, as the microchannel volume can be as small as 2.0-2.5 μL. The microchannels are also less complex to manufacture than other devices that use multiplanar electrodes, as the microchannels described herein have uniplanar electrodes. For example, the microchannel configurations depicted in FIG. 3B and FIG. 6 can be fabricated with the use of a PCB.

As discussed herein, having uniform electric current density throughout a microchannel, or having a uniform distribution of electric current throughout a microchannel, refers to having an electric current that is generally evenly distributed throughout the microchannel, with few or no cold spots where the current density is substantially lower than it is in the surrounding areas.

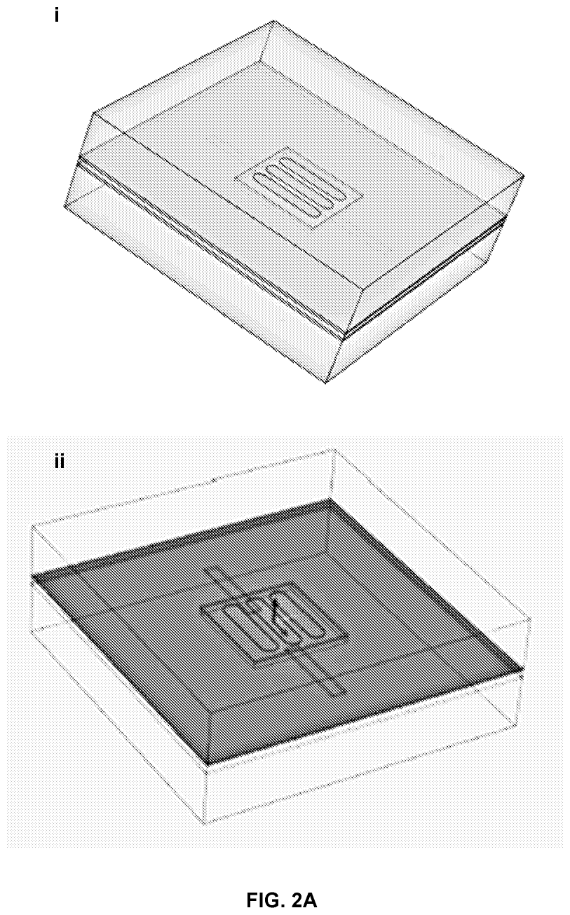

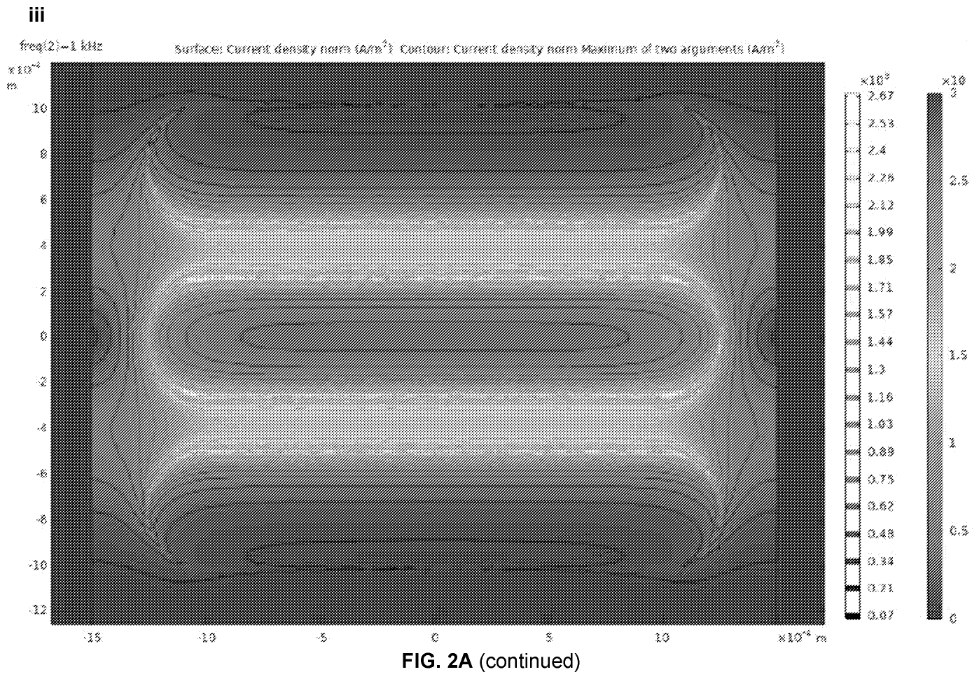

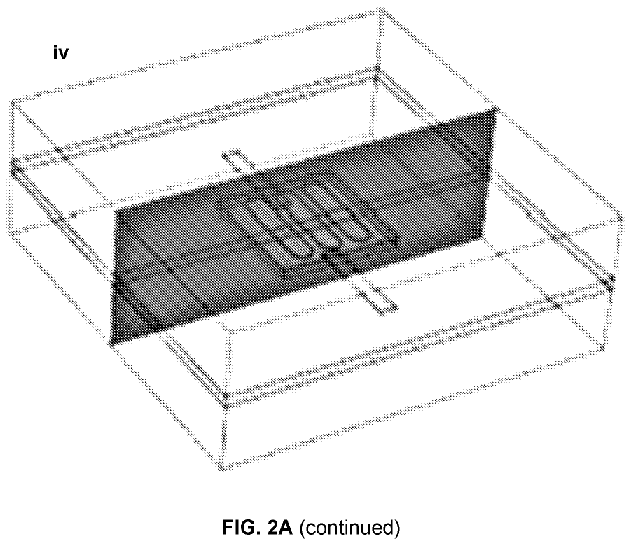

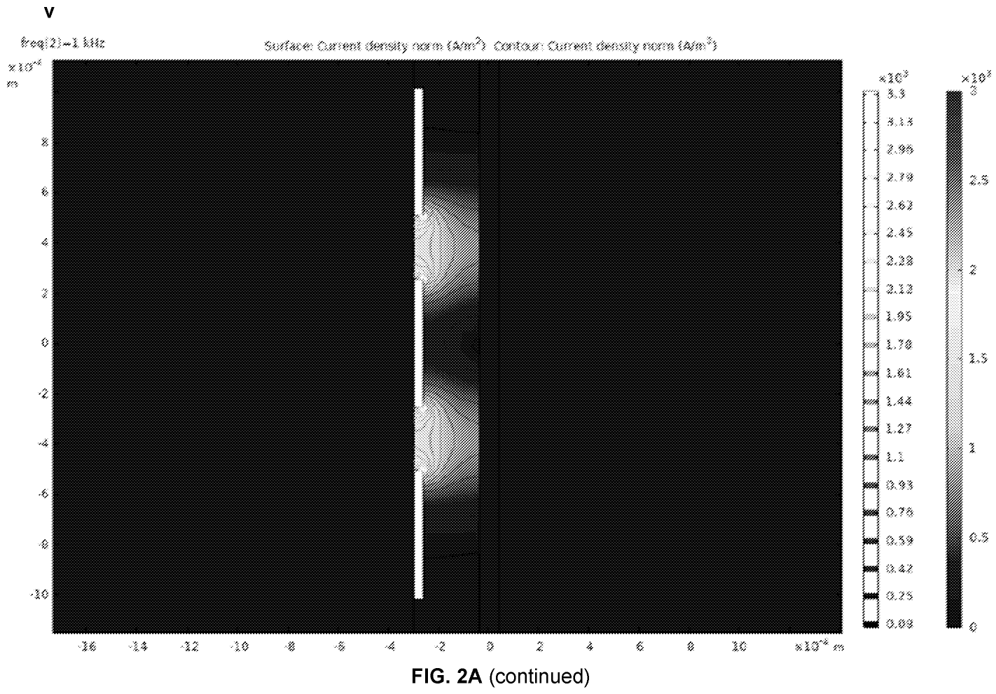

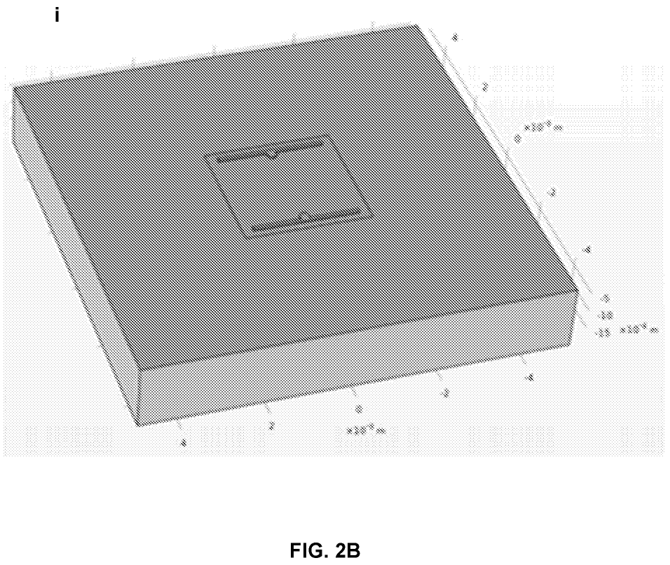

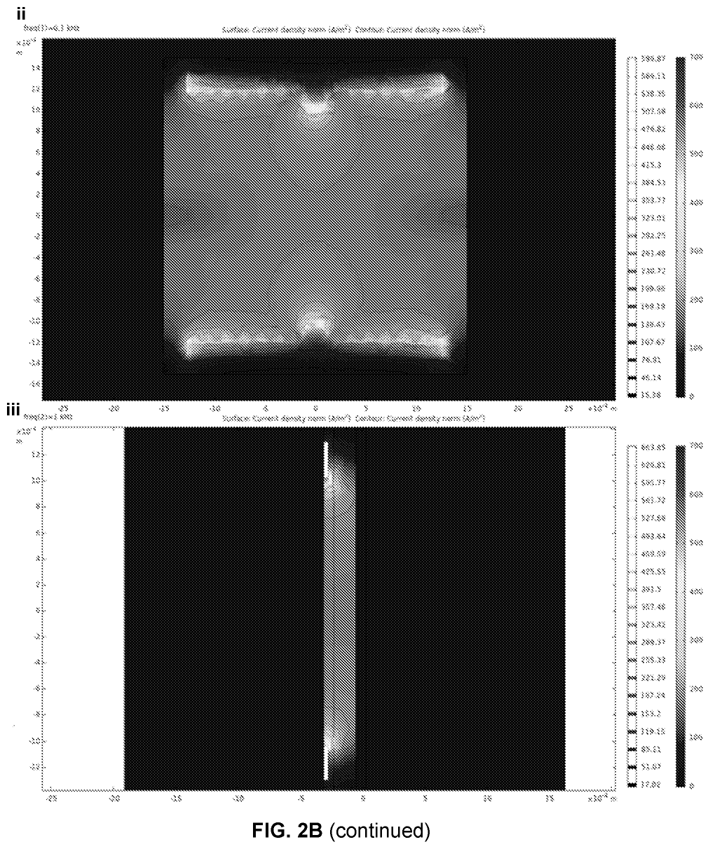

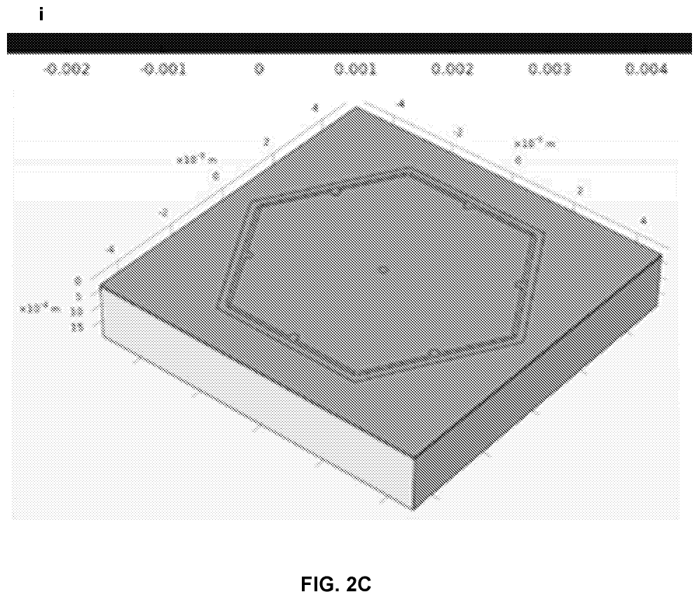

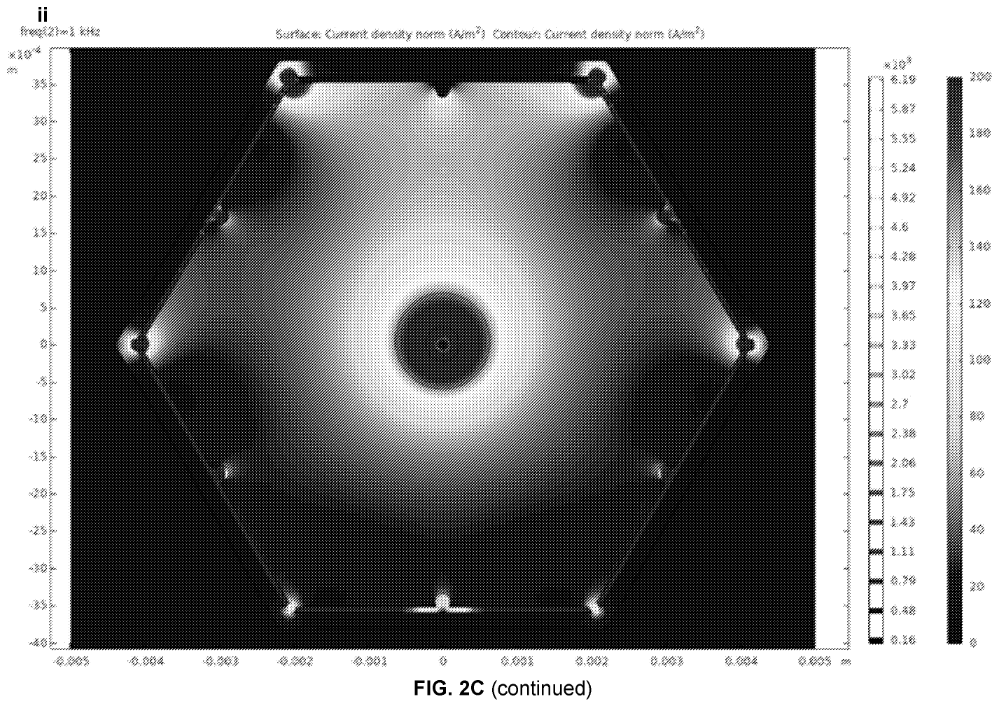

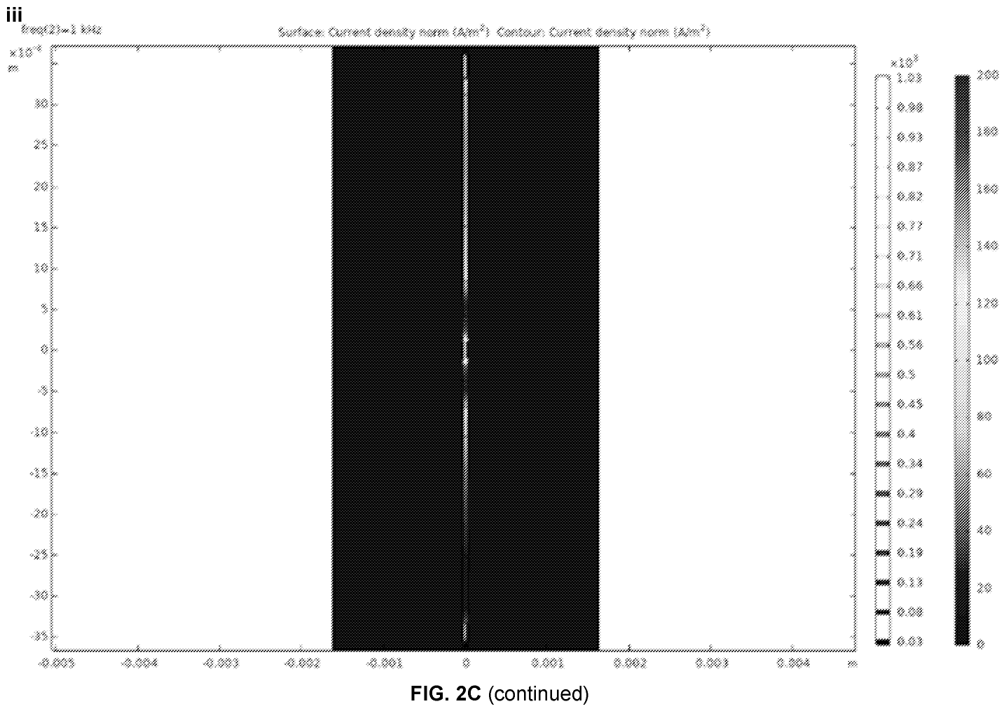

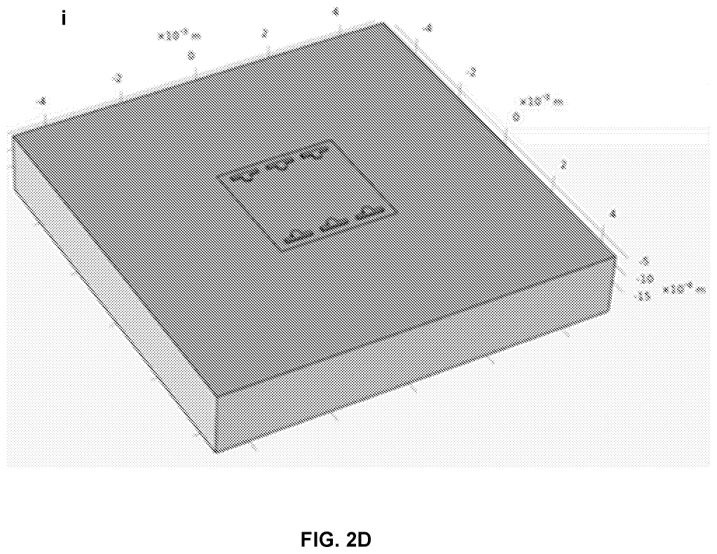

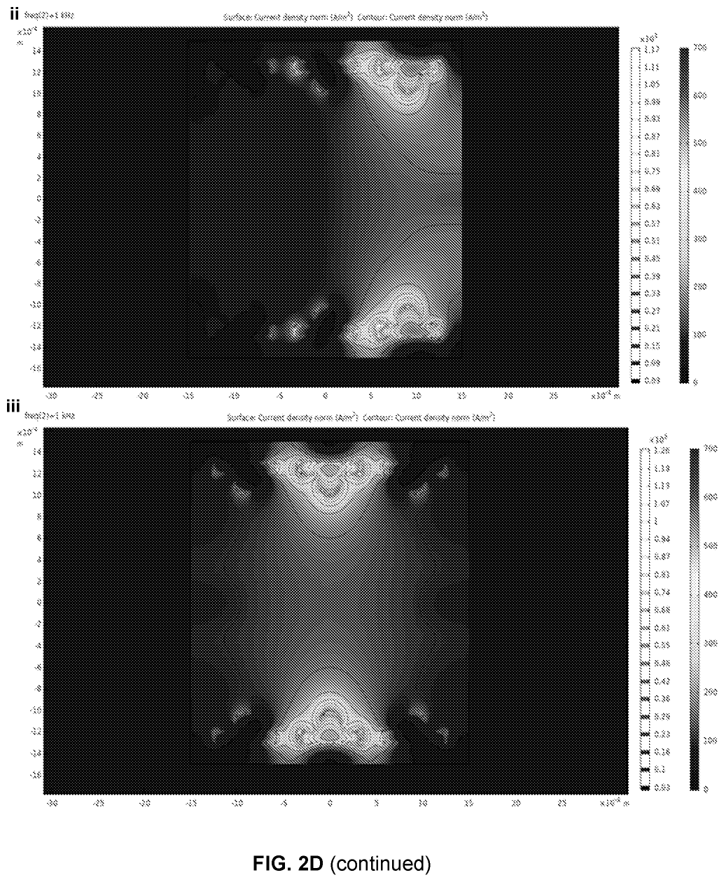

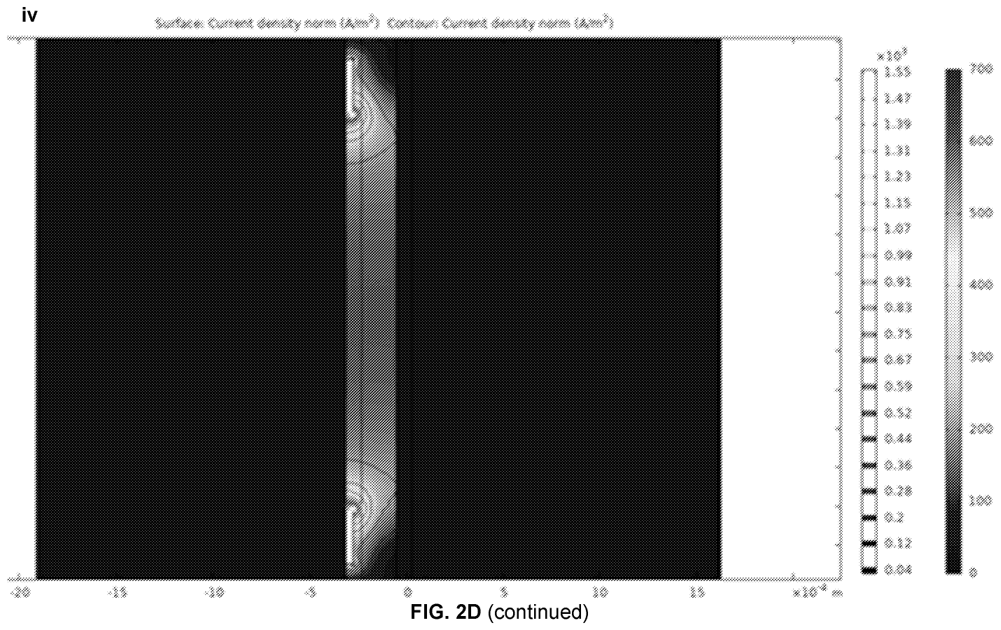

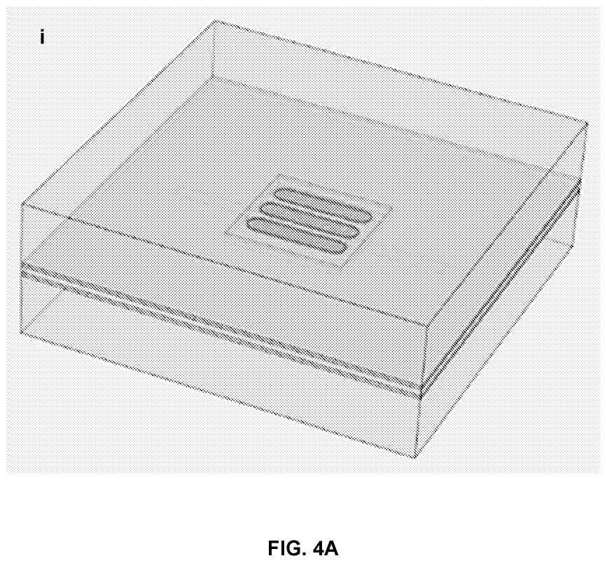

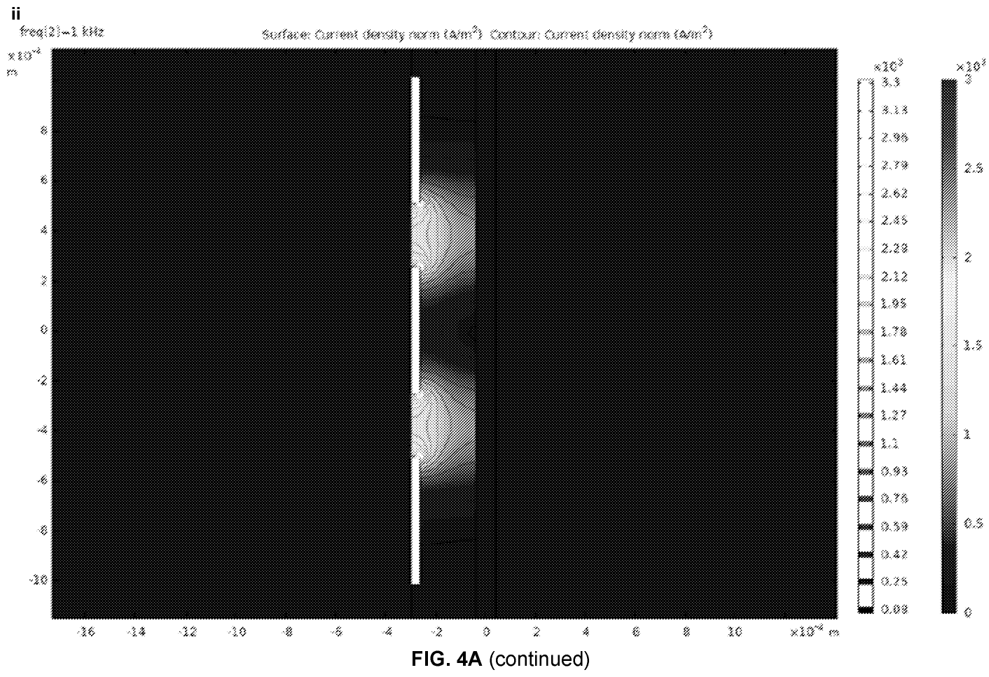

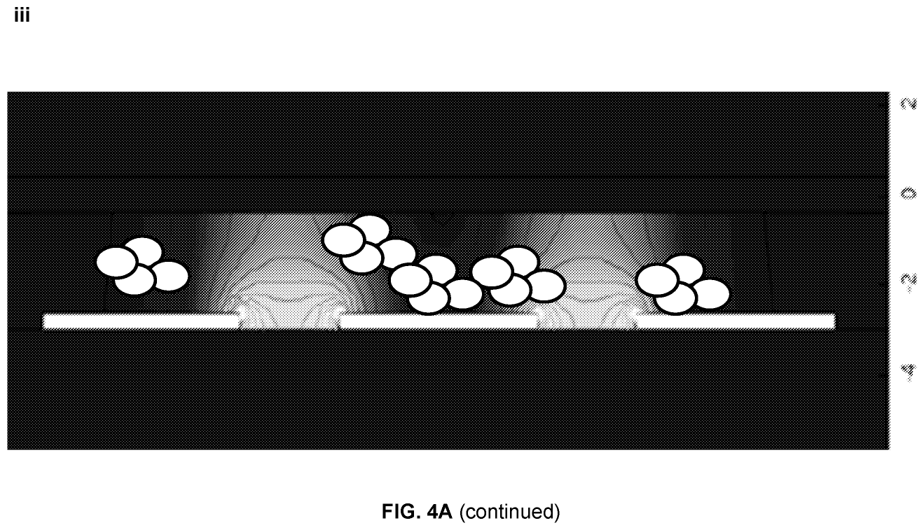

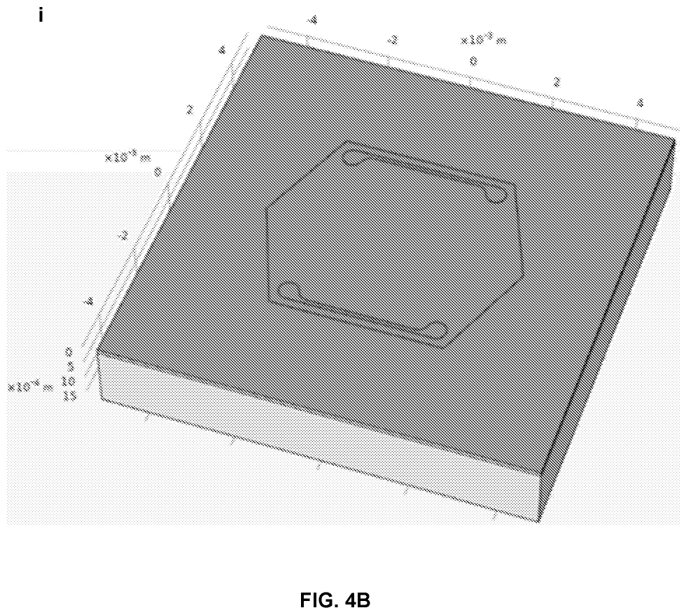

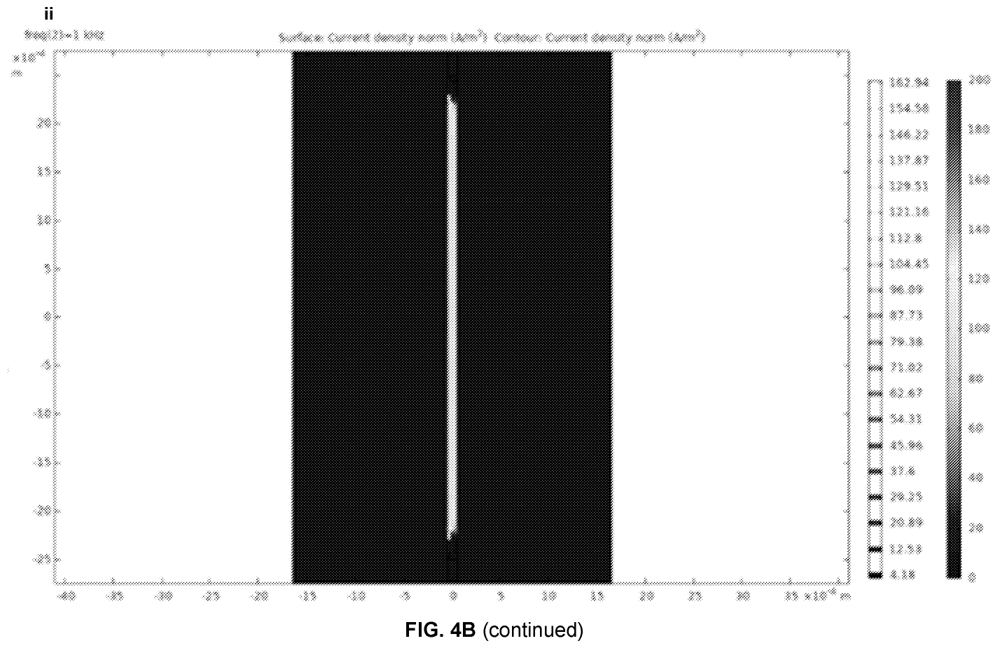

Embodiments of the present invention provide configurations of the electrodes, with respect to microchannel geometry, to achieve the advantages described herein. The relationship between the electrode and microchannel geometry can be critical, especially when the objective is to detect the formation of a blood clot on a minimally thrombogenic and minimally conductive substrate (which, in the Examples described herein, is the floor of the microchannel). For example, as demonstrated herein, when evaluating the distribution of electric current within various microchannel-electrode configurations, the distribution of current, and in turn the ability to detect changes in the current in the microchannel, are biased based on the positions of the electrodes. For example, as demonstrated in FIG. 2B and FIG. 4A, which depict interdigitated electrode configurations, when voltage is applied between the middle electrode and each outer electrode, electric current travels between the middle and both outer electrodes but does not travel to the other areas of the microchannel. Another example is the configuration depicted in FIG. 2C, which has six electrodes (one electrode in the center of the hexagon, and one electrode spanning each edge of the hexagon). In the simulation for this configuration, voltage was applied between the center and one of the six outer electrodes at a given time. The simulation performed with this configuration demonstrates how the electric current preferentially flows to the nearest outer electrode (which is the nearest conductive material), rather than to the central electrode. For configurations such as those discussed above, if a blood clot is formed in an area of the channel that has relatively low current density (e.g., blue and purple areas shown in the figures), the resolution of clot detection and the sensitivity of clot detection will be poor (see, e.g., FIG. 4).

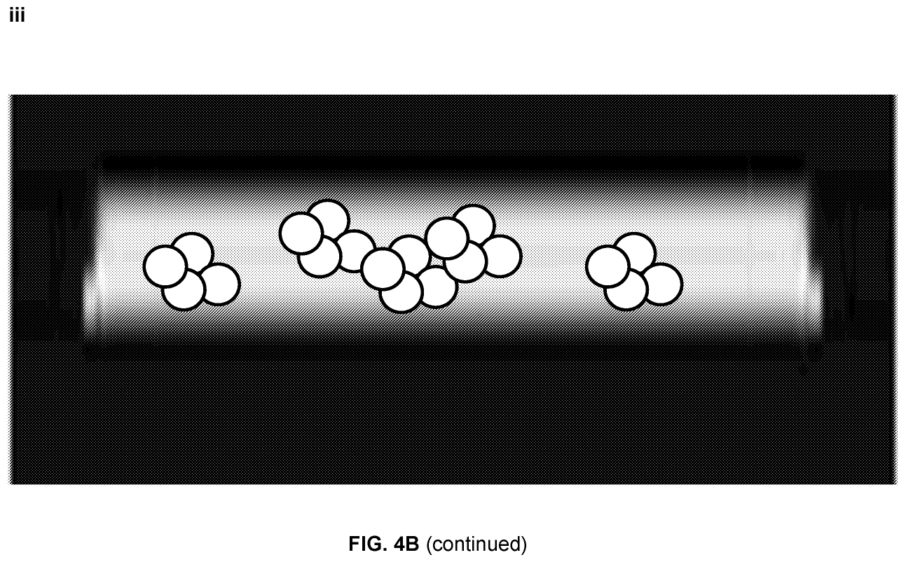

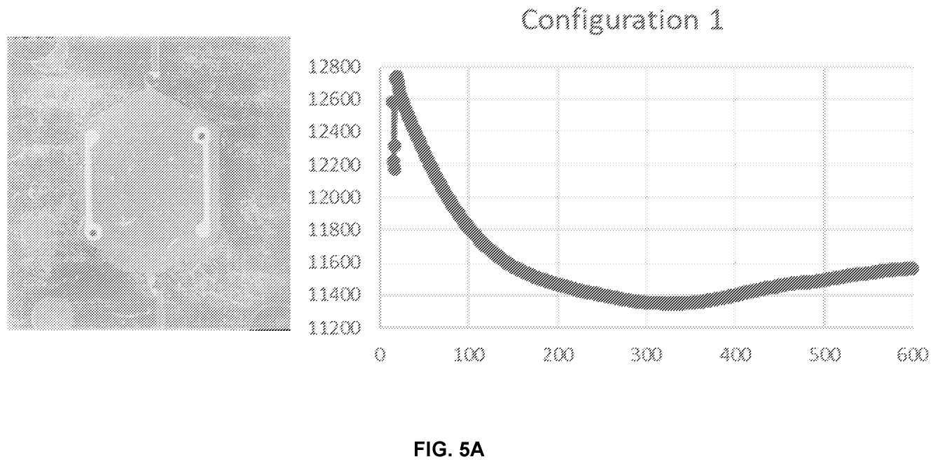

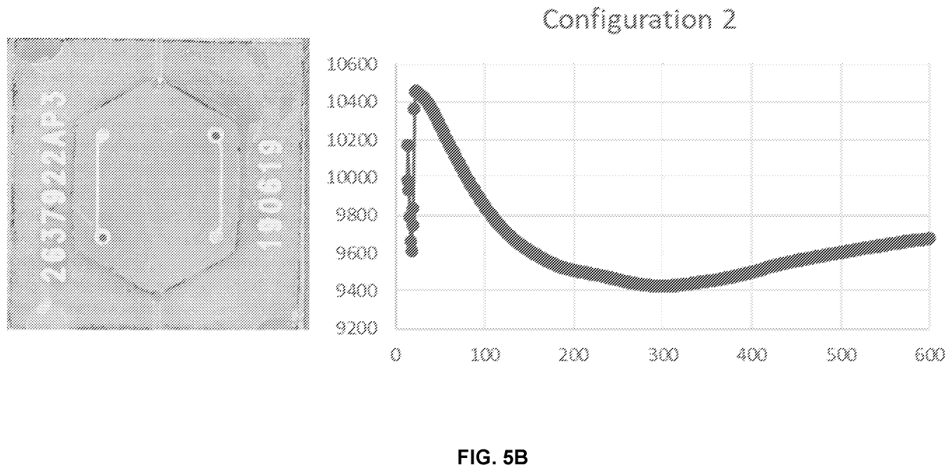

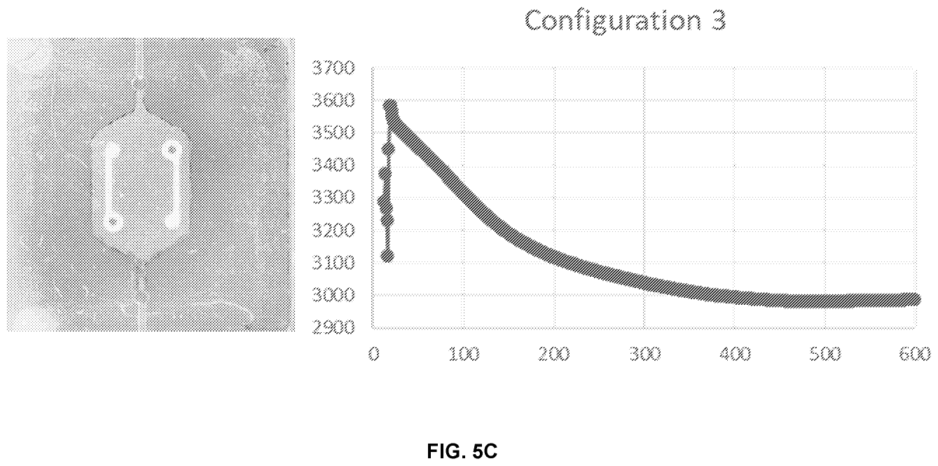

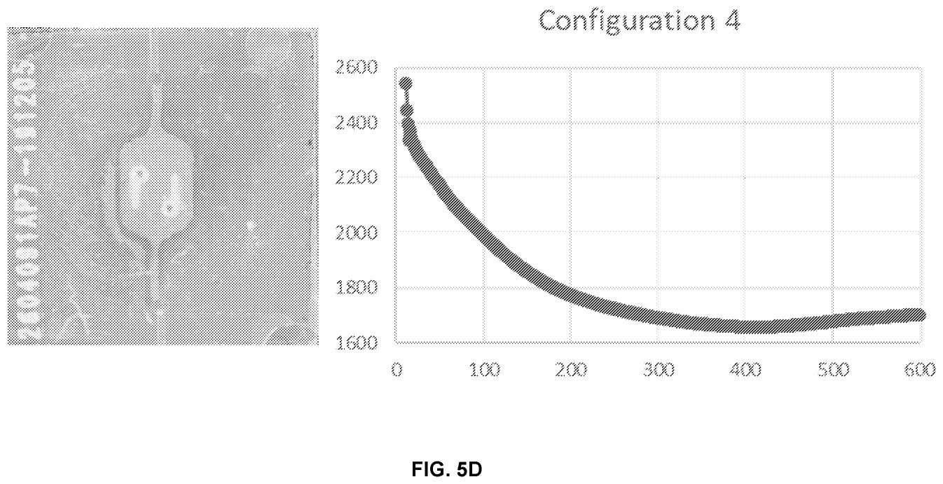

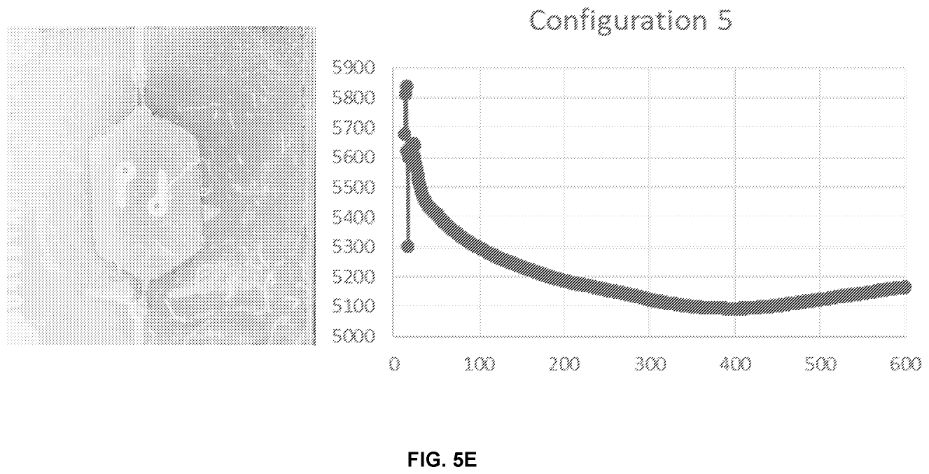

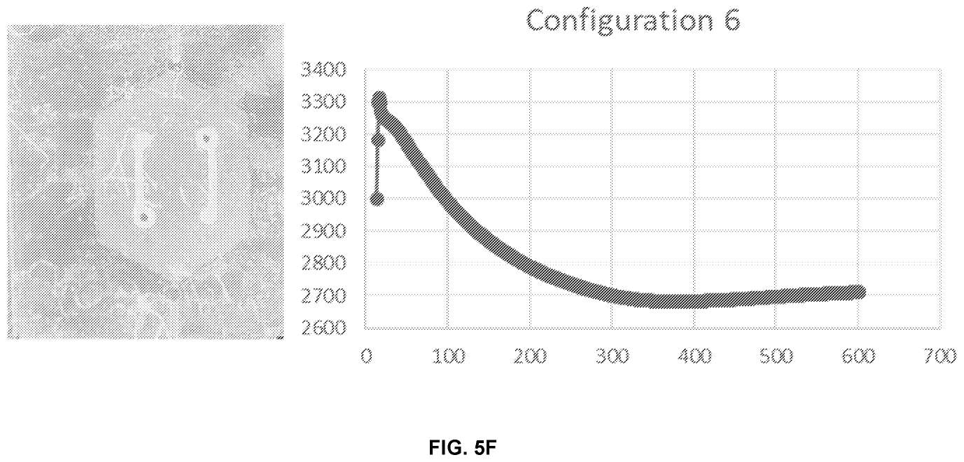

In addition, in the hexagonal two-electrode microchannel configuration, for example, while the electrodes may have uniform electric current distribution between them, if the microchannel extends beyond the electrodes, laterally or dorsally, such extension may reduce the uniformity of current density within the microchannel. The Examples provided herein demonstrate this possible outcome. In Example 4, electrical impedance clot detection was performed on a variety of configurations for a hexagonal two-electrode microchannel (see Table 1 and FIG. 5). Generally, an electrode:channel height ratio that is greater than 0.5 allows for sensitive clot detection (e.g., see Configurations 1 and 2). In Example 5, simulations of electric current distribution were performed for the configuration shown in FIG. 6A, with various channel heights. While an electrode:channel height ratio of 0.0875 exhibited a relatively evenly distributed current density (see FIG. 6B(ii)), an electrode:channel height ratio of 0.35 provided a uniform distribution of current density, at a comparatively high density level (see FIG. 6B(i)).

The distribution of current within the microchannel can also be affected by the distance between electrodes, relative to the width of the microchannel. Spacing the electrodes closer together and farther from the channel's edges may, in some instances, result in poorer clot detection (e.g., see Configuration 5 in FIG. 5 and Table 1).

As demonstrated herein, by using an electrode configuration with a particular microchannel geometry and appropriate electrode:channel height ratio and electrode spacing, a uniform electric current distribution within the microchannel was achieved, allowing for strong clot detection, even when using a minimally thrombogenic substrate for the electrodes and for the surfaces of the microchannel.

The microchannel-electrode configurations provided herein can be used for applications other than assessing blood coagulation; they are useful for any application where detecting or measuring a change in an electrical property in a substance within the microchannel is desired. The configurations also can be used to produce a uniform electric current in a substance within the microchannel, even where there is no need to detect or measure a change in an electrical property. For example, in certain scenarios, it may be desirable to apply an electric current to a substance, and to do so uniformly across the substance. The present invention provides microchannel-electrode configurations, having uniplanar electrodes, that permit such a uniform application of electric current.

While the present invention provides microchannel-electrode systems that do not require a thrombogenic substrate to successfully detect clot formation, it should be understood that thrombogenic substrates may be used in any of the various embodiments of microchannel-electrode configurations described herein. A thrombogenic substrate has surface properties that activate coagulation when the material is in contact with blood. Example thrombogenic substrates include, e.g., glass and metals such as gold.

In addition, in some embodiments, the electrodes can comprise a thrombogenic material. For example, in certain embodiments, the electrodes may have a coating, or may otherwise be made with a material, that comprises a thrombogenic material. Thrombogenic materials that can be used to coat an electrode include, e.g., gold, silver, platinum, and other metals that stimulate coagulation when in contact with blood, as well as non-metals such as collagen. Using electrodes comprising a thrombogenic material can promote localization of clot formation to the area of the electrodes, which can increase the sensitivity of clot detection.

The following examples serve only to illustrate the invention and its practice. The examples are not to be construed as limitations on the scope or spirit of the invention.

### EXAMPLES

Example 1: Simulations of electric current generation were conducted for various configurations of microchannel-electrode configurations, as shown in FIGS. 2A-2D. Each configuration produced a different distribution of electric current density in the simulations. However, none of these configurations provided a uniform distribution of electric current throughout the microchannel. While the configuration depicted in FIG. 2B shows relatively uniform electric current density in the microchannel, the electric current is concentrated at the two poles and at the center of each electrode. Simulations were performed using COMSOL Multiphysics 5.4 with the AC/DC Module (COMSOL, Inc., Burlington, MA, USA). The properties of the medium inside the microchannels approximated unclotted blood (electrical conductivity: 0.703 S/m; relative permittivity: 5120).

FIG. 2A depicts an interdigitated configuration. The excitation voltage was 0.6 V AC between each of the two outer electrodes and middle electrode. FIG. 2B depicts a two-electrode configuration. The excitation voltage was 0.6 V AC between the two electrodes. FIG. 2C depicts a hexagonal multiplexed configuration. An excitation voltage of 0.6 V AC was applied between the center electrode and one of the six edge electrodes at a given time. This configuration reflects an attempt to improve spatial resolution of clot detection within a microchannel by measuring a small portion of the microchannel at a time. However, the inactive electrodes diverted current away from the medium and reduced uniformity of current density. FIG. 2D depicts a multiplexed configuration with three electrode pairs. An excitation voltage of 0.6 V AC was applied between one of the three electrode pairs in the microchannel, as another attempt to improve spatial resolution of detecting current changes. However, this configuration, like the configuration in FIG. 2C, produced poor uniformity in current density.

For all figures depicting current density (FIG. 2A(ii)(v), FIG. 2B(ii)(iii), FIG. 2C(ii)(iii), FIG. 2D (ii)-(iv), FIG. 3A (ii)(iii), FIG. 3B(ii)(iii), FIG. 4A(ii), FIG. 4B(ii), and FIG. 6B(i)-(v)), the two vertical legends to the right of the microchannel diagram show current density (A/m²). The first (left-hand) vertical legend provides values for the contour plot, while the second (right-hand) vertical legend provides values for the surface plot.

Example 2: Simulated electric current generation was compared for microchannels having the same geometry but with different electrode configurations, as shown in FIGS. 3A-3C. While the electrodes in FIG. 3A and FIG. 3B have the same height (35 μm) and width (100 μm), in FIG. 3B, the location of the electrical vias was changed (relative to the location in FIG. 3A). In addition, each electrode in FIG. 3B has two circular or bulb regions, one circular region at each electrode end, whereas each electrode in FIG. 3A has one circular region (which contains the via and is located at the electrode's center). In FIG. 3B, the circular region at each electrode end has a diameter of 0.5 mm (see also FIG. 3C). These differences in electrode geometry greatly affected the distribution of current in the microchannel (compare FIG. 3A(ii) to FIG. 3B(ii)). By changing the location, size, and number of the circular regions in each electrode in the microchannel, the uniformity of electric current within the microchannel is improved, as shown in FIG. 3B; in addition, the level of electric current density is generally higher (e.g., compare current density levels in the center regions of FIG. 3A(ii) and FIG. 3B(ii)). A comparison of these configurations demonstrates how changing the electrode geometry, for example at the electrode poles, can affect the electric current distribution within the microchannel. Simulations were performed using COMSOL Multiphysics 5.4 with the AC/DC Module (COMSOL, Inc., Burlington, MA, USA). The properties of the medium inside the microchannels approximated unclotted blood (electrical conductivity: 0.703 S/m; relative permittivity: 5120). The excitation voltage was 0.6 V AC between the two electrodes in each case.

Example 3: Simulated electric current density was compared for two microchannel-electrode configurations, as shown in FIGS. 4A-4B. FIG. 4A depicts an interdigitated configuration, and FIG. 4B depicts a two-electrode configuration. Simulations were performed using COMSOL Multiphysics 5.4 with the AC/DC Module (COMSOL, Inc., Burlington, MA, USA). The properties of the medium inside the microchannels approximated unclotted blood (electrical conductivity: 0.703 S/m; relative permittivity: 5120). The excitation voltage was 0.6 V AC between the two outer electrodes and middle electrode in FIG. 4A (interdigitated configuration) and between the two electrodes in FIG. 4B (two-electrode configuration). The schematics in FIG. 4A(iii) and FIG. 4B(iii) illustrate how a more uniform distribution of electric current improves clot detection. In FIG. 4A(iii), the clots (represented by the aggregates of white circles) in the blue areas (areas of low electric current density) are less likely to be detected; to detect a clot in this configuration, the clot must be large enough to overlap with a higher-current green area, and thus clot detection depends on clot location within the microchannel and on clot size. In contrast, the more uniform current distribution in FIG. 4B(iii) increases the likelihood of early and successful detection of clot formation within the microchannel, as detection depends less on the location of clot formation.

Example 4: Six configurations were evaluated for their ability to detect clot formation based on changes in electrical impedance. The configurations are shown in FIG. 5 and are described in Table 1. The electrodes used in each configuration were gold-plated electrodes. Each electrode included one via (a connection to the other side of the circuit board that interfaces with the external electronics), which is circular in shape and is located at one pole (end) of the electrode. The circular via is surrounded by a circular region or bulb in the electrode. For certain configurations, the circular geometry was mimicked at the other end of the electrode-see configurations b 1, 2, 3, and 6 in FIG. 5. Using electrodes having the circular geometry at both electrode ends promotes symmetrical current distribution, whereas using electrodes having the circular geometry at only one end can result in a colder spot towards the end without the circular geometry, as that portion of the electrode is farther from the opposing electrode than is the electrode end having the circular geometry.

A clot is detected when the slope of impedance changes from negative to positive. In Table 1, the time corresponding to this change in slope is provided as “Time of clot detection.” As a cross-linked fibrin clot continues to form, electrical impedance increases until it peaks. The graphs in FIG. 5 show how electrode geometry and placement, and microchannel geometry, affect how well the electrodes can sense changes in electrical impedance and in turn detect clot formation. While all configurations demonstrated successful clot detection, the time of the clot detection, which is an indicator of the sensitivity of the microchannel-electrode system, differs across the configurations. For example, configuration 2 (FIG. 5B) detects a clot at 300 seconds, while configuration 3 (FIG. 5C) does not detect a clot until about 480 seconds. An additional parameter is the change in impedance that occurs as the clot forms; a greater increase in impedance, starting from the time when the slope of impedance changes from negative to positive, indicates stronger and more sensitive clot detection, which may be due to increased sensitivity to, and a better ability to detect, changes in cell membrane capacitance that accompany coagulation. For example, configuration 1 (FIG. 5A) exhibited a strong clot detection signal, with an impedance change of>200 ohms, whereas configuration 6 (FIG. 5F) exhibited a comparatively poor clot signal, with an impedance change of<100 ohms. Configuration 2 (FIG. 5B) also exhibited sensitive clot detection.

Example 5: Simulations of electric current generation were performed for a two-electrode configuration to assess the effect of channel height on current distribution, as shown in FIGS. 6A-6B. Simulations were performed using COMSOL Multiphysics 5.4 with the AC/DC Module (COMSOL, Inc., Burlington, MA, USA). The properties of the medium inside the microchannels approximated unclotted blood (electrical conductivity: 0.703 S/m; relative permittivity: 5120). The excitation voltage was 0.6 V AC between the two electrodes at a frequency of 10 kHz. The current density (A/m²) in the medium along the indicated plane (see FIG. 6A(iii)) was plotted for microchannels with different heights ranging from 100 μm to 2400 μm (100, 200, 300, 400, 600, 800, 1,200, 1,600, 2,400 μm). All other channel dimensions were held constant and are shown in FIG. 6A(ii) (length=6 mm, edge=4 mm, width=5 mm, electrode spacing=4.5 mm). The electrode height was 35 μm. The electrode spacing distance (4.5 mm) is the distance between the linear portions of the electrodes, excluding the round ends. FIG. 6B provides the current density plots for microchannel heights of 100 μm, 400 μm, 1,200 μm, 1,600 μum, and 2,400 μm. The distribution of current becomes less uniform as microchannel height increases. With increasing microchannel height, the current is given more volume within which to spread, leading to a lower current density farther from the electrodes, and less uniformity of current density overall.

In addition, a range of excitation frequencies between 100 Hz and 1 MHz were used in simulations. Varying frequency exhibited a negligible effect on current distribution, indicating that the impedance between electrodes is largely resistive in this model. The 10 kHz frequency is shown for all cases in FIG. 6B.

TABLE 1
Example configurations for a microchannel with two electrodes

| | | | | | | | | Time of | |
|---|---|---|---|---|---|---|---|---|---|
| | Channel | Channel | Channel | Channel | Electrode | | Electrode | clot | Clot |
| | width | length | height | volume | height | Electrode:Channel | spacing | detection | detection |
| Configuration | (mm) | (mm) | (μm) | (μL) | (μm) | height ratio | (mm) | (seconds) | assessment |
| 1 | 5.5 | 5.8 | 64 | 2.04 | 62 | 0.97 | 4.14 | 320 | Good |
| 2 | 4.8 | 5.25 | 78 | 1.97 | 46 | 0.59 | 4.05 | 300 | Good |
| 3 | 3.25 | 4.25 | 148 | 2.04 | 46 | 0.31 | 2.25 | 480 | Poor |
| 4 | 2.25 | 3.0 | 296 | 2.00 | 46 | 0.16 | 1.25 | 410 | Poor |
| 5 | 4.8 | 5.25 | 78 | 1.97 | 46 | 0.59 | 1.25 | 400 | Poor |
| 6 | 3.25 | 4.25 | 148 | 2.04 | 46 | 0.31 | 2.25 | 385 | Poor |

TABLE 2
Electrical Conductivity

| Material | Electrical Conductivity (S/m) | |
|---|---|---|
| Polyimide | 5.26 | × 10⁻¹⁶ |
| Polycarbonate | 7.94 | × 10⁻¹⁵ |
| Acrylic | 2.86 | × 10⁻¹⁴ |
| Silicone | 1.00 | × 10⁻¹³ |
| Glass | 1.00 | × 10⁻¹³ |
| FR-4 | 2.50 | × 10⁻¹¹ |
| Silicon | 4.35 | × 10⁻⁴ |
| Blood | 7.00 | × 10⁻¹ |
| Gold | 4.11 | × 10⁷ |
| Copper | 5.96 | × 10⁷ |

Unless otherwise expressly specified, the numerical ranges and values recited herein, such as the values for ratios of dimensions, may be read as if prefaced by the word “about” even though the term “about” may not expressly appear with the numerical range or value. At the very least, and not as an attempt to limit the application of the doctrine of equivalents to the scope of the claims, each numerical value should be construed in light of the number of reported significant digits and by applying ordinary rounding techniques. In addition, when numerical ranges are set forth herein, these ranges are inclusive of the recited range end points (e.g., end points may be used).

While this invention has been particularly shown and described with references to preferred embodiments thereof, it will be understood in light of the present disclosure by persons skilled in the art that various changes in form and details may be made therein without departing from the scope of the invention encompassed by the appended claims.

What is claimed is:

1. A microchannel comprising two electrodes positioned on a single plane of the microchannel, wherein the geometry of the microchannel is a hexagon with six edges, and wherein each of the two electrodes comprises a rectangular region, each electrode's rectangular region being parallel to one of the microchannel's six edges and parallel to the other electrode's rectangular region, and wherein one or more of the following features is present:
a. the electrode height:channel height ratio is at least 0.1;
b. the electrode spacing:channel width ratio is at least 0.5;
c. each of the two electrodes comprises two circular regions, each circular region being located at an electrode end.

2. The microchannel according to claim 1, wherein the electrode height:channel height ratio is 0.3 or greater.

3. The microchannel according to claim 1, wherein the electrode height:channel height ratio is 0.5 or greater.

4. The microchannel according to claim 1, wherein the electrode height:channel height ratio is 0.6 or greater.

5. The microchannel according to claim 1, wherein the electrode height:channel height ratio is 0.8 or greater.

6. The microchannel according to claim 1, wherein each of the two electrodes comprises two circular regions, each circular region being located at an electrode end, and wherein each circular region has a diameter that is 500 microns.

7. The microchannel according to claim 1, wherein each of the two electrodes comprises two circular regions, each circular region being located at an electrode end, and wherein each circular region has a diameter that is within the range of 300 microns to 600 microns.

8. The microchannel according to claim 7, wherein each circular region has a diameter that is 600 microns.

9. The microchannel according to any one of claims 1-8, wherein the volume of the microchannel is 2.5 microliters or lower.

10. The microchannel according to any one of claims 1-8, wherein the volume of the microchannel is 2.0 microliters or lower.

11. The microchannel according to any one of claims 1-8, wherein the volume of the microchannel is 2.0-2.5 microliters.

12. The microchannel according to any one of claims 1-11, wherein the interior surface of the microchannel is minimally thrombogenic.

13. The microchannel according to any one of claims 1-12, wherein the interior surface of the microchannel is minimally conductive.

14. The microchannel according to any one of claims 1-13, wherein each of the two electrodes provides a surface that is more thrombogenic than all other surfaces within the microchannel.

15. The microchannel according to any one of claims 1-14, wherein the floor of the microchannel comprises polyimide.

16. The microchannel according to any one of claims 1-15, wherein each of the two electrodes comprises a thrombogenic coating.

17. The microchannel according to claim 16, wherein the thrombogenic coating comprises one or more of gold, silver, and platinum.

18. The microchannel according to any one of claims 1-17, wherein the electrode height:channel height ratio is at least 0.1; the electrode spacing:channel width ratio is at least 0.5; and each of the two electrodes comprises two circular regions, each circular region being located at an electrode end.

19. A hexagonal microchannel comprising two electrodes oriented to be opposite one another and on the same plane of the hexagonal microchannel, each electrode comprising a rectangular region that is parallel to one of the hexagonal microchannel's six edges and that is located no more than 500 microns from the edge to which it is parallel, wherein the distance between the electrodes' rectangular regions is at least 4,000 microns, and wherein the length of each edge is no longer than 4,000 microns.

20. The hexagonal microchannel according to claim 19, wherein the electrode height:channel height ratio is 0.3 or greater.

21. The hexagonal microchannel according to any one of claims 19-20, wherein the interior surface of the hexagonal microchannel is minimally thrombogenic.

22. The hexagonal microchannel according to any one of claims 19-21, wherein the interior surface of the hexagonal microchannel is minimally conductive.

23. The hexagonal microchannel according to any one of claims 19-22, wherein the floor of the hexagonal microchannel comprises polyimide.

24. The hexagonal microchannel according to any one of claims 19-23, wherein each of the two electrodes comprises a thrombogenic coating.

25. The hexagonal microchannel according to claim 24, wherein the thrombogenic coating comprises one or more of gold, silver, and platinum.
